(19) United States
(12) Patent Application Publication
(10) Pub. No.: US 2020/0282191 A1
(43) Pub. Date: Sep. 10, 2020

(54) INFLATION DEVICE WITH SELF ALIGNING CRANK HANDLE

(71) Applicant: Merit Medical Systems, Inc., South Jordan, UT (US)

(72) Inventor: Gregory R. McArthur, Sandy, UT (US)

(21) Appl. No.: 16/809,212

(22) Filed: Mar. 4, 2020

Related U.S. Application Data
(60) Provisional application No. 62/814,521, filed on Mar. 6, 2019.

(51) Int. Cl. A61M 25/10

(52) U.S. Cl. CPC A61M 25/10182; A61M 25/10184

(57) ABSTRACT

Devices used to pressurize, depressurize, or otherwise displace fluid are disclosed. The devices may be configured to displace fluid in order to inflate or deflate a medical device, such as a balloon. The devices include a crank member for providing a mechanical advantage when pressurizing or otherwise displacing fluid. A self-aligning grip is included for facilitation closure of the crank member.

### RELATED APPLICATIONS

The present application claims priority to U.S. Provisional Application 62/814,521, filed on Mar. 6, 2019, and titled, “Inflation Device with Self Aligning Crank Handle,” which is hereby incorporated by reference in its entirety.

### TECHNICAL FIELD

The present disclosure relates generally to devices used to pressurize, depressurize, or otherwise displace fluid, for example in medical devices. More specifically, the present disclosure relates to high-pressure devices used to pressurize, depressurize, or otherwise displace fluid along a line in order to inflate or deflate a medical device, such as a balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of an inflation device assembly.

FIG. 1B is a perspective view of the inflation device assembly of FIG. 1A, shown in a partially deployed state.

FIG. 1C is a perspective view of a portion of the inflation device assembly of FIG. 1A, shown in a deployed state.

FIG. 2 is a perspective view of a grip of the inflation device assembly of FIG. 1A.

FIG. 3 is a perspective view of a portion of the inflation device assembly of FIG. 1A shown in a partially deployed state.

FIG. 4 is a partial cutaway, partial schematic, view of a portion of the inflation device assembly of FIG. 1A in a partially deployed state.

### DETAILED DESCRIPTION

An inflation device may comprise a syringe which utilizes threads to advance or retract a plunger by rotating the plunger handle relative to the body of the syringe such that the threads cause longitudinal displacement of the plunger relative to the body. In some instances, an inflation syringe may comprise retractable threads, configured to enable a practitioner to disengage the threads and displace the plunger by simply pushing or pulling the plunger.

The inflation syringe may comprise a coupling member configured to constrain movement of the plunger within the syringe body. The coupling member may comprise threads configured to engage with the retractable threads. Certain inflation devices include a mechanism in the handle of the device which allows the practitioner to disengage the threads through manipulating the mechanism. For example, in some instances the handle of such a device may include a “trigger” portion configured to retract threads positioned on the plunger. Actuation of the trigger may thus transition the threads between an engaged configuration where the threads are engaged with the coupling member and a released, or disengaged configuration, where the plunger is configured to be displaced with respect to the syringe body by pushing or pulling on the plunger.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Further, various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the syringe portion of an inflation device, the proximal end of the syringe refers to the end nearest the handle and the distal end refers to the opposite end, the end nearest the inlet/outlet port of the syringe. Thus, if at one or more points in a procedure a physician changes the orientation of a syringe, as used herein, the term “proximal end” always refers to the handle end of the syringe (even if the distal end is temporarily closer to the physician).

“Fluid” is used in its broadest sense, to refer to any fluid, including both liquids and gases as well as solutions, compounds, suspensions, etc., which generally behave as fluids.

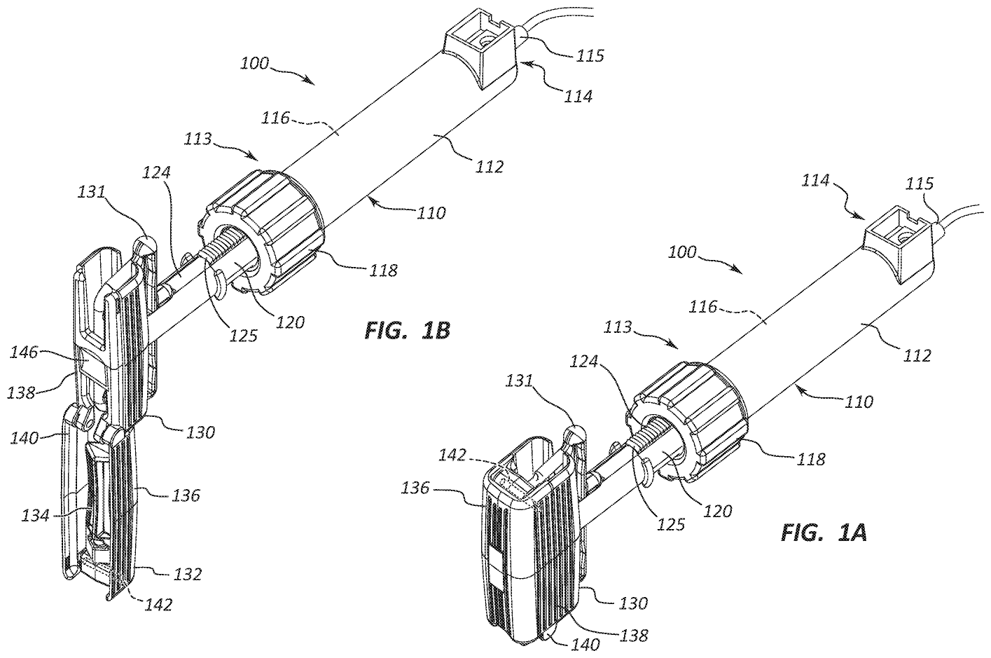

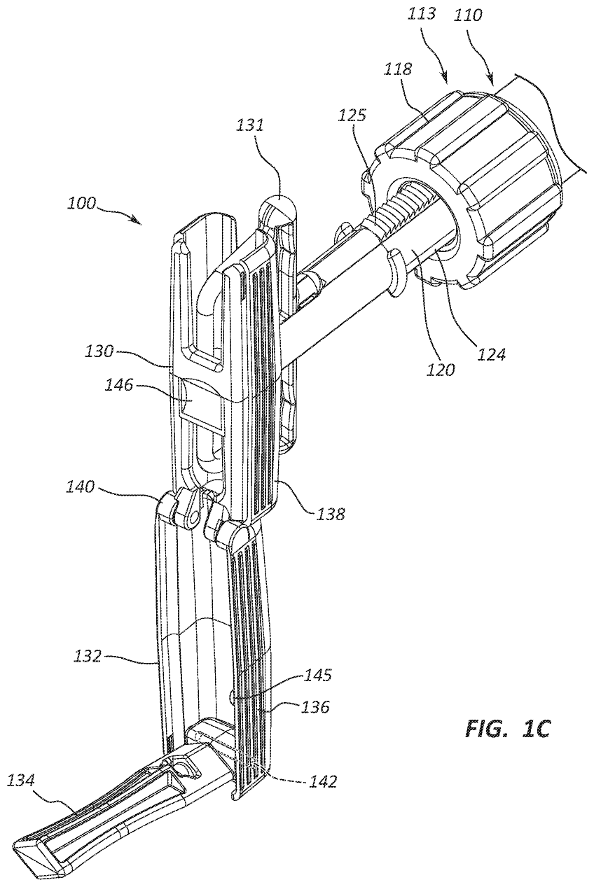

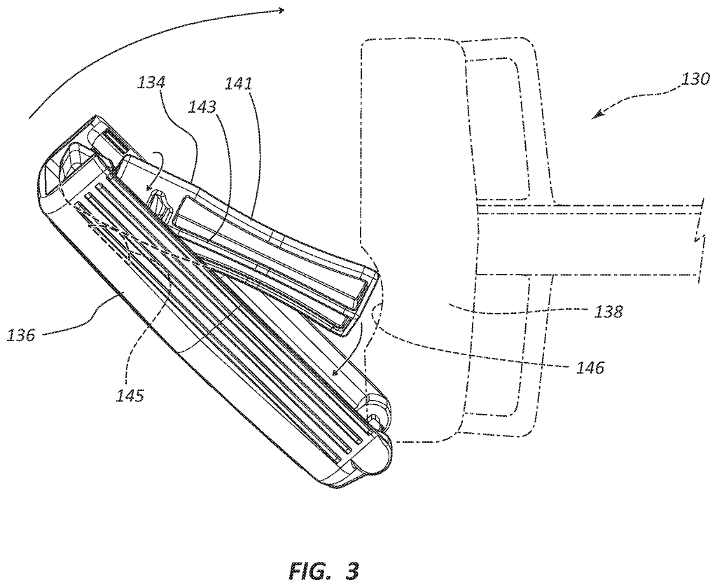

FIGS. 1A-4 illustrate different views of an inflation device and related components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1A-1C depict an embodiment of an inflation device assembly 100. In the illustrated embodiment, the inflation device assembly 100 comprises a syringe 110. The inflation device assembly 100 may be described as comprising three broad groups of components; each of these groups may have multiple subcomponents and parts. The three broad component groups are: a body component such as syringe body 112, a pressurization component such as a plunger 120, and a handle 130.

The syringe body 112 may be formed of a generally cylindrical hollow tube configured to receive the plunger 120. The syringe body 112 may include an inlet/outlet port 115 located adjacent the distal end 114 of the syringe body 112. In some embodiments, a coupling member 118 may be coupled to the syringe body 112 adjacent a proximal end 113 of the syringe body 112. The coupling member 118 may include a center aperture configured to allow the plunger 120 to pass through the coupling member 118 into the syringe body 112. Further, the coupling member 118 may include coupling member threads configured to selectively couple the coupling member 118 to the plunger 120. In some embodiments, the coupling member 118 comprises a polymeric nut at the proximal end 113 of the syringe body 112.

The plunger 120 may be configured to be longitudinally displaceable within the syringe body 112. The plunger 120 may be comprised of a plunger shaft coupled to a plunger seal at the distal end of the plunger shaft. The plunger shaft may also be coupled to the handle 130 at the proximal end of the plunger shaft, with the plunger shaft spanning the distance between the plunger seal and the handle 130.

The handle 130 broadly refers to the group of components coupled to the proximal end of the plunger 120, some of which may be configured to be graspable by a user. In certain embodiments, the handle 130 may be configured such that the user may manipulate the position of the plunger 120 by manipulating the handle 130. Further, in some embodiments, the handle 130 may be an actuator mechanism configured to manipulate components of the inflation device 100.

The components disclosed in connection with any of the exemplary handle configurations herein may be optional. That is, though the handle 130 broadly refers to the components coupled to the proximal end of the plunger shaft which may be configured to be graspable by a user, use of the term “handle” is not meant to indicate that every handle component present in every embodiment within the scope of this disclosure. Rather, the term is used broadly, referring to the collection of components, but not specifically referring to or requiring the inclusion of any particular component, such as the crank member 132. Likewise, other broad groupings of components disclosed herein, such as the syringe 110 or syringe body 112 and the plunger 120, may also refer to collections of individual subcomponents. Use of these terms is also non-limiting, as each subcomponent may or may not be present in every embodiment.

Furthermore, the inflation device assembly 100 described herein may be configured for use with a crank handle, such as crank member 132. The inflation device assembly 100 may be configured for use both with the crank member 132 deployed or with the crank member 132 in an undeployed state. In other words, systems within the scope of this disclosure may be configured to displace a plunger (through direct longitudinal displacement or through rotation of threads) with the crank member 132 undeployed. Thus, the system may be configured such that a practitioner has the option of deploying the crank member 132, or utilizing the system in a manner similar to conventional systems, with the crank member 132 undeployed.

As shown in FIGS. 1A-1B, a fluid reservoir 116 may be defined by the space enclosed by the inside walls of the syringe body 112 between the plunger seal and the distal end 114 of the syringe body 112. Accordingly, movement of the plunger seal with respect to the syringe body 112 alters the size and volume of the fluid reservoir 116.

As shown in FIGS. 1A-1C, in some embodiments, the syringe 110 may comprise a coupling member 118, fixedly coupled to the proximal end 113 of the syringe body 112. The coupling member 118 may utilize threads or other coupling mechanisms to fixedly couple the coupling member 118 to corresponding threads on the syringe body 112. Additionally, the coupling member 118 may be configured to engage external plunger threads 125 configured to couple the plunger 120 to the coupling member 118. When the plunger threads 125 and the coupling member 118 are engaged, the plunger 120 may be translated longitudinally with respect to the syringe body 112 by rotating the plunger 120 such that the interaction of the coupling member threads on the inside diameter of the coupling member and the plunger threads 125 results in the longitudinal translation of the plunger 120. Such rotating motion may be achieved when a practitioner grasps and rotates the handle 130. In some embodiments, clockwise rotation may be configured to extend the plunger 120 distally and counter-clockwise rotation may be configured to retract the plunger 120 proximally. Other embodiments may be configured with reverse threads configured to displace the plunger distally when rotated counterclockwise and proximally when rotated clockwise.

Thus, when the plunger threads 125 and the coupling member threads are engaged, movement of the plunger 120 is constrained with respect to the syringe body 112, though the plunger 120 is not necessarily fixed with respect to the syringe body 112. For example, the plunger 120 may be rotatable, but not directly translatable, when the threads are engaged.

The plunger threads 125 may be configured such that they may be retracted within the plunger shaft. In some embodiments, the plunger threads 125 do not extend 360 degrees around the axis of the plunger shaft. For example, the plunger threads 125 may be formed on a thread rail 124 on the plunger shaft. The thread rail 124 may be retracted from the threads of the coupling member 118 by actuating a mechanism such as a trigger 131.

The retractable threads may allow a practitioner to displace the plunger 120 relative to the syringe body 112 either through rotation of the plunger 120 (and the subsequent interaction of threads), or by retracting the plunger threads 125 and displacing the plunger 120 by applying opposing forces on the plunger 120 and the syringe body 112. (The forces may move the plunger 120 distally or proximally with respect to the syringe body 112.) Both methods of displacement may be utilized during the course of a single therapy.

In some instances, a practitioner may desire to quickly displace the plunger 120, for instance, while priming the inflation device or while priming or deflating an attached medical device, such as a balloon. Quick displacement of the plunger 120 may be accomplished by retracting the plunger threads 125 and sliding the plunger 120 relative to the syringe body 112. For example, a practitioner may quickly fill the fluid reservoir 116 with fluid by disengaging the plunger threads 125 and pulling the plunger 120 in a proximal direction with respect to the syringe body 112. Further, a practitioner may quickly force fluid into lines leading to a medical device or quickly expel unwanted air bubbles from the fluid reservoir 116 by retracting the plunger threads 125 and repositioning the plunger 120.

In other instances, the practitioner may desire more precise control over the position of the plunger 120 (for example when displacing the plunger 120 in order to adjust the fluid pressure within the fluid reservoir 116) or it may simply be difficult to displace the plunger 120 due to high fluid pressure within the fluid reservoir 116. In these instances, the practitioner may opt to displace the plunger 120 by rotation of the plunger 120.

When a practitioner rotates the handle 130 the plunger 120 may be advanced distally or retracted proximally through the threaded engagement of the thread rail 124 and the coupling member 118. At high pressures, it can be difficult to rotate the handle 130 in order to increase the corresponding pressure in the medical device. In the embodiment depicted in FIGS. 1A-1C, the handle 130 comprises a crank member 132 that is extendable from the handle 130, for example, in a cantilevered fashion. The crank member 132 may further comprise a grip 134 for grasping by the practitioner's hand or fingers. The grip 134, in turn, may be hingedly coupled to the crank member 132. Rotation of the handle 130 using the crank member 132 when positioned in an extended or deployed position may thus generate additional mechanical advantage due to the offset of the grip 134 from the axis of rotation (the axis of the plunger shaft) which provides leverage to further advance the plunger 120 at high internal pressures.

Referring to FIGS. 1A-1C: FIG. 1A shows the inflation device assembly prior to deployment of the crank member 132 with the crank member 132 in axial alignment with the handle 130; FIG. 1B shows the inflation device assembly 100 with the crank member 132 in a partially deployed state such that the crank member 132 extends laterally from the handle 130 and the grip 134 is nested within a cavity or channel of the crank member 132; FIG. 1C shows a portion of the inflation device assembly 100 with the crank member 132 fully deployed such that the crank member 132 extends laterally from the handle 130 and the grip 134 is elevated to a vertical orientation.

The crank member 132 and grip 134 can be disposed in a deployed state for use as depicted in FIG. 1C and an undeployed state nested within the handle 130 as depicted in FIG. 1A. In other words, the crank member 132 may be nested within the handle 130 and may comprise an integrated part of the handle 130. The crank member 132 may comprise a top portion 136 of the handle 130 that is hingedly coupled to a bottom portion 138 of the handle 130. The crank member 132 may be rotatable about a first hinge 140 in order to transition the crank member 132 from an undeployed state to a deployed state. When in the deployed state, the crank member 132 may extend in a direction substantially perpendicular to the longitudinal axis of the syringe 110 and plunger 120. The grip 134 may also be rotatable about a second hinge 142. When deployed, the grip 134 may extend in a direction substantially parallel to (but radially offset from) the longitudinal axis of the syringe 110 and plunger 120.

In the undeployed state, the grip 134 may be disposed within a channel or cavity of the crank member 132, which, in turn, functions as the top portion 136 of the handle 130. Thus, the grip 134 may be nested or disposed within the handle 130 and may be concealed within the handle 130. The practitioner may selectively advance or retract the plunger 120 using the handle 130 when the crank member 132 is in the undeployed state (similar to conventional systems) or when the crank member 132 is in the deployed state utilizing the additional mechanical advantage generated by the crank member 132.

The crank member 132 may thus be configured to provide additional leverage in advancing the plunger 120 to achieve elevated pressures with the inflation device assembly 100, while also permitting disengagement of the thread rail 124 from the coupling member 118 to rapidly move the plunger 120 longitudinally within the syringe body 112. For example, once high inflation pressures are achieved in the inflation device 100 using the crank member 132, deflation of the balloon can be achieved rapidly through actuating the trigger 131 to disengage the thread rail 124 and not requiring a cranking motion to retract the plunger 120. Furthermore, the grip 134 may be configured to provide an ergonomic interface with the crank member 132 for the practitioner to facilitate rotation of the handle 130.

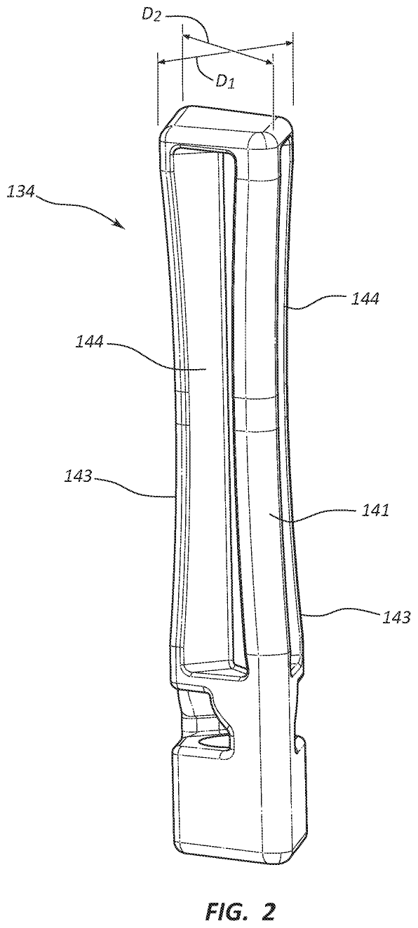

As illustrated in FIG. 2, the grip 134 may be a rhomboid shape in cross-section. As also noted above, the grip 134 may be configured to be nested in the cavity or channel of the crank member 132 prior to deployment of the crank member 132. The grip 134 may include a cross-sectional first width D₁ that is larger than a cross-sectional second width D₂. The first width D₁ may be about 25% to about 100% larger than the second width, D₂, including about 30% to about 70% larger, and about 50%. In some embodiments, the grip 134 may comprise longitudinal first ribs 141, second ribs 143, and/or recesses 144 which may be configured to facilitate its gripability. In the illustrated embodiment, the first ribs 141 are aligned with the first width D₁ and the second ribs 143 are aligned with the second width D₂. In other embodiments, the grip 134 may comprise other grip enhancing features such as bumps, texturing, soft material covering, etc. The grip 134 may be configured to axially rotate relative to the crank member 132 as the crank member 132 is rotated by the practitioner such that the practitioner does not ungrasp and re-grasp the grip 134 as the crank member 132 is rotated.

The grip 134 may be configured to be rotationally self-orienting such that the grip 134 self-orients to a low profile orientation when the crank member 132 is displaced from the deployed state to the undeployed state. Such alignment may be configured to allow the handle 130 to maintain a low profile when the crank member 132 is disposed in the undeployed state as shown in FIG. 1A. FIG. 1B illustrates the grip 134 nested in the cavity of the crank member 132 in the low profile orientation. In the low profile orientation, the first width D₁ of the grip 134 is oriented horizontally and the second width D₂ is oriented vertically.

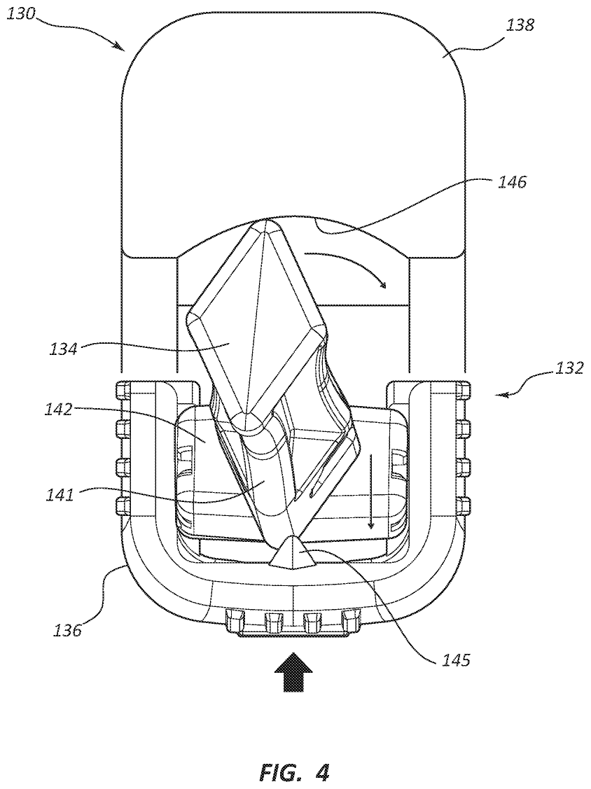

FIG. 4 illustrates the grip 134 in a partially deployed state. When the grip is displaced from the deployed state to the undeployed state, the first rib 141 of the grip 134 may be configured to engage with internal walls of the cavity or channel of the crank member 132 to rotationally self-orient the grip 134 to the low profile orientation. If the first rib 141 is not aligned with a longitudinal axis of the crank member 132 as it is displaced, it may be rotationally deflected by a bottom wall of the cavity as illustrated in FIG. 4 as a rotational force vector is directed to the first rib 141. The rotational deflection of the first rib 141 may continue until the grip 134 is disposed in the low profile orientation and the crank member 132 is in the undeployed state. The rotational deflection may be directed to either side of the cavity of the crank member 132.

As shown in FIGS. 1C, 3 and 4, the crank member 132 may include a deflecting rib 145 disposed adjacent the second hinge 142. The deflecting rib 145 may extend longitudinally within the cavity or channel of the crank member 132 and may be offset from a longitudinal axis of the crank member 132. The deflecting rib 145 may have a triangular shape. When the grip 134 is displaced from the deployed state to the undeployed state, the deflecting rib 145 may be configured to engage with a proximal end of a first rib 141 if the first rib 141 is axially aligned with the longitudinal axis of the crank member 132. This axial alignment may prevent the grip 134 from self-orienting to a low profile orientation in embodiments without the deflecting rib 145 due to a lack of the rotational force vector. When engaged, the deflecting rib 145 may be configured to apply a rotational force vector causing rotational deflection of the first rib 141 such that the first rib 141 rotates from the axially aligned orientation to a non-axially aligned orientation as depicted in FIG. 4. This allows the grip 134 to rotate to the low profile orientation as the crank member 132 is disposed to the undeployed state.

As depicted in FIGS. 1B, 1C, and 4, the bottom portion 138 of the handle 130 may comprise an axial load depression 146 configured to axially load the grip 134 when the crank member 132 is disposed from the deployed state to the undeployed state. The axial load depression 146 may be cup-shaped and configured to engage with a distal end of the first and second ribs 141, 143. This engagement results in the grip 134 being axially loaded such that an axial load is exerted on the second hinge 142. In other embodiments that do not include the axial load depression 146, a grip may be laterally loaded as a crank member is disposed from a deployed state to an undeployed state. This lateral loading may exert a sheer load on a second hinge that couples the grip to the crank member resulting in breakage of the second hinge, in some instances.

In use the inflation devices and systems described above may be pressurized using any of the following steps or actions, each of which may be optional or interchanged. An inflation device is obtained which comprises a syringe body, a plunger within the syringe body, a handle coupled to the plunger (such as through a thread rail coupled to a coupling member) and a crank member coupled to the handle. The crank member may be deployed from a nested position in the handle prior to rotation of the crank member.

The plunger may be advanced by grasping the syringe body in one hand, and grasping a grip and rotating the crank member with the other hand. Before rotation of the crank member, the thread rail of the plunger may be disengaged from the syringe body (or coupling member). The plunger may be advanced through longitudinal movement of the handle to a first internal pressure. Then the thread rail may be re-engaged to the syringe body after reaching the first internal pressure. The plunger may be further advanced through rotational movement of the handle via the crank member to achieve a second pressure. After the therapy is complete or when desirous of depressurizing the syringe, the thread rail can be disengaged from the syringe body and retracted through longitudinal movement of the handle.

The crank member may be undeployed by folding the grip into the crank member. The grip may self-orient to a low profile orientation when folded. The grip may also be loaded at a distal end when engaging with the handle and exert an axial load on a hinge.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An inflation device assembly, comprising:
a syringe body;
a plunger configured for advancement and retraction within the syringe body; and
a coupling member comprising coupling member threads configured to constrain movement of the plunger within the syringe body;
a handle coupled to a proximal portion of the plunger; and
a crank member coupled to the handle;
wherein the crank member comprises a grip configured to rotationally self-orient when the crank member is moved from a deployed state to an undeployed state.

2. The inflation device assembly of claim 1, wherein the grip comprises a rhomboid shape in a transverse cross-sectional plane, wherein the grip comprises a first width that is larger than a second width.

3. The inflation device assembly of claim 2, wherein when the grip self-orients, the first width is oriented in a horizontal plane and the second width is oriented in a vertical plane.

4. The inflation device assembly of claim 1, wherein the grip is disposed within a cavity or channel of the crank member when the crank member is disposed in the undeployed state.

5. The inflation device assembly of claim 1, wherein the crank member comprises a deflecting rib configured to rotationally deflect the grip when the grip is moved from the deployed state to the undeployed state.

6. The inflation device assembly of claim 5, wherein the deflecting rib is disposed off-center from a longitudinal axis of the crank member.

7. The inflation device assembly of claim 1, wherein the handle comprises a depression configured to axially load the grip when the grip is moved from the deployed state to the undeployed state.

8. The inflation device assembly of claim 7, wherein the depression comprises a cup shape.

9. The inflation device assembly of claim 1, wherein the crank member is hingedly coupled to the handle.

10. A rotatable handle, comprising:
a bottom member; and
a crank member hingedly coupled to the bottom member;
wherein the crank member comprises a grip configured to rotationally self-orient when the crank member is moved from a deployed state to an undeployed state.

11. The rotatable handle of claim 10, wherein the grip comprises a rhomboid shape in a transverse cross-sectional plane, where a first width is larger than a second width.

12. The rotatable handle of claim 11, wherein the grip self-orients such that the first width is oriented in a horizontal plane and the second width is oriented in a vertical plane.

13. The rotatable handle of claim 10, wherein the grip is disposed within a cavity or channel of the crank member when the crank member is disposed in the undeployed state.

14. The rotatable handle of claim 10, wherein the grip is configured to facilitate a user's grasp of the crank member with the user's hand or fingers.

15. A method of pressurizing a medical device, comprising:
obtaining an inflation device comprising a syringe body, a plunger within the syringe body, a handle coupled to the plunger, and a crank member coupled to the handle, wherein the crank member comprises a grip;
deploying the crank member;
grasping the syringe body in one hand and the grip in the other hand; and
advancing the plunger by rotating the crank member.

16. The method of claim 15, further comprising disposing the crank member from a deployed state to an undeployed state.

17. The method of claim 16, wherein the grip is configured to rotationally self-orient when disposed in the undeployed state.

18. The method of claim 15, wherein the crank member comprises a deflecting rib configured to rotationally deflect the grip when disposed in the undeployed state.

19. The method of claim 15, wherein the grip is configured to be axially loaded when disposed in the undeployed state.

20. The method of claim 15, wherein the handle comprises an axial load depression configured to axially load the grip when disposed in the undeployed state.
